SEQUENCE LISTING (4 sequences)

SEQ ID NO: 1 - DNA, 327 bp, Homo sapiens
atgaagttcatctcgacatctctgcttctcatgctgctggtcagcagcctctctccagtccaaggtgttctggaggtctattacacaagcttgaggtgtagatgtgtccaagagagctcagtctttatccctagacgcttcattgatcgaattcaaatcttgccccgtgggaatggttgtccaagaaaagaaatcatagtctggaagaagaacaagtcaattgtgtgtgtggaccctcaagctgaatggatacaaagaatgatggaagtattgagaaaaagaagttcttcaactctaccagttccagtgtttaagagaaagattccc

SEQ ID NO: 2 - DNA, 783 bp, Homo sapiens
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactc

SEQ ID NO: 3 - protein, 109 aa, Homo sapiens
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 4 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

(19) United States
(12) Patent Application Publication
(10) Pub. No.: US 2021/0030795 A1
(43) Pub. Date: Feb. 4, 2021

(54) ALLOGENEIC T-CELL-BASED HIV VACCINE TO INDUCE CELLULAR AND HUMORAL IMMUNITY

(71) Applicant: Enochian BioPharma, Inc., Los Angeles, CA (US)

(72) Inventor: Serhat Gumrukcu, Los Angeles, CA (US)

(21) Appl. No.: 16/904,319

(22) Filed: Jun. 17, 2020

Related U.S. Application Data
(60) Provisional application No. 62/862,432, filed on Jun. 17, 2019.

(51) Int. Cl. A61K 35/17; C12N 15/87; C07K 14/705; C07K 14/52; C07K 14/16

(52) U.S. Cl. CPC A61K 35/17; C12N 15/87; C07K 14/16; C07K 14/52; C07K 14/70575

(57) ABSTRACT

Provided herein are methods for treating a patient with human immunodeficiency virus (HIV), comprising administering cellular compositions comprising recombinant allogeneic cells, such as CD4+ T cells. The present invention further relates to compositions and methods for making an allogeneic T-cell-based protective HIV vaccine that induces both cellular and humoral immunity. Related compositions and methods for modulating the immune system using such recombinant cells are also provided.

### CROSS-REFERENCE TO RELATED APPLICATIONS

This application claims priority to U.S. Provisional Application No. 62/862,432, filed Jun. 17, 2019, which is hereby incorporated by reference in their entirety.

### FIELD OF THE INVENTION

Provided herein are methods for treating a patient with human immunodeficiency virus (HIV), comprising administering cellular compositions further comprising recombinant allogeneic cells, such as CD4+ T cells. The present invention further directs to compositions and methods for making an allogeneic T-cell-based protective HIV vaccine that induces both cellular and humoral immunity.

### BACKGROUND OF THE INVENTION

Significant progress has been made against the HIV epidemic. However, currently there are still 1.7 million new infections per year and 770,000 deaths worldwide. Antiretroviral therapy (ART), which is the most successful method of treatment, can cost $18,000 to $40,000 per year. The total expended on HIV from 2000-2016 was $562 billion dollars and is almost $50 billion per year. Thus, a vaccine and cure are badly needed.

### SUMMARY

In certain embodiments, provided herein is a cell comprising a heterologous nucleic acid molecule comprising a nucleotide sequence encoding CD40L and CXCL13.

In additional embodiments, the CD40L comprises an amino acid sequence having at least 90% sequence identity to SEQ ID NO: 4, and wherein the CXCL13 comprises an amino acid sequence having at least 90% sequence identity to SEQ ID NO: 3.

In further embodiments, the cell is a T cell.

In further embodiments, the cell is transduced or transfected with a second and/or third nucleic acid that encodes a heterologous protein.

In further embodiments, the second nucleic acid comprises human immunodeficiency virus (HIV) genome, and wherein the HIV genomic nucleic acid comprises a mutation in the retroviral reverse transcriptase, and further wherein the HIV genomic nucleic acid does not encode a retroviral packaging signal, creating a disabled HIV genomic construct.

In certain embodiments, provided herein is a CD4+ cell comprising one or more heterologous nucleic acid molecules encoding for CD40L and CXCL13.

In further embodiments, the CD40L comprises an amino acid sequence having at least 90% sequence identity to SEQ ID NO: 4, and wherein the CXCL13 comprises an amino acid sequence having at least 90% sequence identity to SEQ ID NO: 3.

In certain embodiments, provided herein is a CD4+ cell comprising a heterologous CD40L protein and heterologous CXCL13 protein.

In further embodiments, the CD4+ cell further comprises heterologous nucleic acid molecule comprising human immunodeficiency virus (HIV) genome, and wherein the HIV genomic nucleic acid comprises a mutation in the retroviral reverse transcriptase, and further wherein the HIV genomic nucleic acid does not encode a retroviral packaging signal, creating a disabled HIV genomic construct.

In certain embodiments, provided herein is a method of treating HIV in a subject comprising administering to the subject a composition comprising administering an effective amount of any of the cells described herein above.

In certain embodiments, provided herein is a method for increasing immune response in a subject in need thereof, comprising administering an effective amount of any of the cells described herein above.

In additional embodiments, the cells are allogeneic to the subject.

In additional embodiments, the cells are not HLA-matched with the patient.

In additional embodiments, the dosage of cells ranges from about 1-5×10⁶.

In additional embodiments, the viral infection is caused by human immunodeficiency virus (HIV).

In additional embodiments, graft versus host disease (GVHD) is decreased or eliminated, while graft versus virus (GVV) is increased in the subject.

In additional embodiments, the treatment or increasing the immune response is repeated periodically for time frames of from once every week, to once every 2 weeks, to once every 3 weeks, to once per month, to once every two months, to once every 3 months, to once every 4 months, to once every 5 months, to once every 6 months, or once every 7 months, or once every 8 months, or once every 9 months, or once every 10 months, or once every 11 months, or once annually as a maintenance treatment for as long as the subject exhibits improvement, decreased or undetectable viral titer, or stable/non-progressing disease.

In additional embodiments, cellular and humoral immunity are induced in the subject.

### BRIEF DESCRIPTION OF THE DRAWINGS

FIG. 1A illustrates a non-limiting vector map for making human and macaque constructs.

FIG. 1B illustrates a non-limiting vector map for making human and macaque constructs.

FIG. 2 illustrates a scheme for using embodiments provided herein macaques.

FIG. 3 illustrates a scheme for using embodiments provided herein.

FIG. 4 illustrates a scheme for using embodiments provided herein

FIG. 5 illustrates data showing enhanced cytotoxicity of immune cell compositions provided herein.

FIG. 6 illustrates increases in NK cells after vaccination of PBMCs with cells provided for herein.

FIG. 7 illustrates increases in NKT cells after vaccination of PBMCs with cells provided for herein.

FIG. 8 illustrates increases in NK cell activation after vaccination of PBMCs with cells provided for herein.

FIG. 9 illustrates increases in NK cell activation humoral response after vaccination of PBMCs with cells provided for herein.

FIG. 10 illustrates increases in B cell activation after vaccination of PBMCs with cells provided for herein.

FIG. 11 illustrates increases in T cell activation after vaccination of PBMCs with cells provided for herein.

FIG. 12 illustrates increases in T cell activation after vaccination of PBMCs with cells provided for herein.

FIG. 13 illustrates increases in CD8 T cell activation after vaccination of PBMCs with cells provided for herein.

### DETAILED DESCRIPTION

Abbreviations:

Antibody-dependent cellular cytotoxicity: ADCC

Cluster differentiation 3: CD3

Graft-versus-tumor effects: GVT

Graft versus host disease: GVHD

Graft versus virus: GVV

Gamma delta T cells: GDT cells, also γδT cells.

Human immunodeficiency virus (HIV): A lentivirus that causes acquired immunodeficiency syndrome.

Invariant natural killer T cells: iNK T cells, also known as type I or classical NKT cells, are a distinct population of T cells that express an invariant αβ T-cell receptor (TCR) and a number of cell surface molecules in common with natural killer (NK) cells.

Natural Killer cells: NK cells

Effective efforts to induce a sufficient immune response to protect against HIV have been limited. Although significant titers of the neutralizing antibody can be achieved and prevent infection in non-human primates, thus far translation of humoral vaccines to human success has been elusive. Vaccines that promote cellular responses have shown promise in animals as several are currently in human trials. However, the data have shown to date that efficacy is limited.

A vaccine that could effectively combine a strong humoral and cellular response, would be a potent approach. Even if such a vaccine failed as a protective vaccine, it could have efficacy as a therapeutic vaccine.

The present invention relates to compositions and methods for making an allogeneic (or in certain embodiments, autologous) T-cell-based protective HIV vaccine that induces both cellular and humoral immunity. The general outline of these methods and compositions include the following:

T cells from a donor or a cell line are infected by a replication incompetent, or live attenuated strain of HIV.

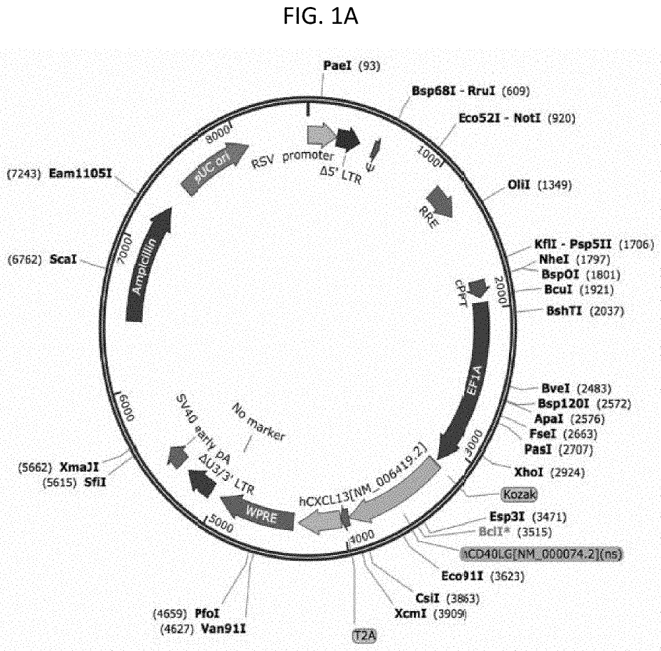

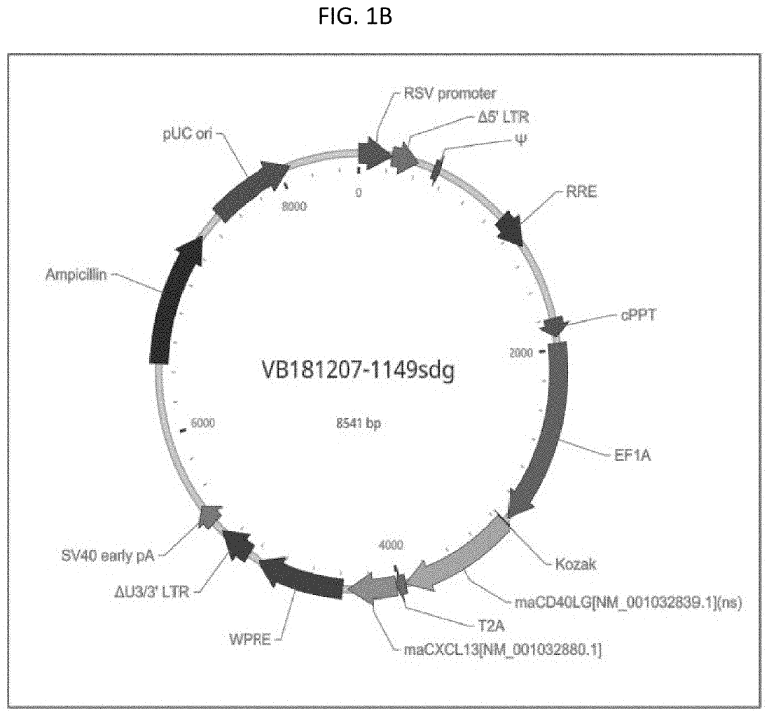

Upon being injected into a host, unless the injected cell population is very high, the host immune system would reject the whole live-attenuated-HIV-infected T cell population easily, only by means of cytotoxic immune response without giving the humoral immune system the opportunity to engage with the allo-T cells during the rejection process. Thus, humoral immunity would not be elicited. To ensure the recruitment of the host B cells into the rejection process, infected allo-T cells are genetically modified, by means of viral or non-viral genetic modification tools, to express human CD40L and human CXCL13. (See FIG. 1A, example of a hCD40L and hCXCL13 expressing vector map).

The expression of hCD40L and hCXCL13 molecules will facilitate a B-cell-specific rejection of the allo-T cells and ensure the humoral immune system's activation against the allo-T cells, as well as the HIV genome that is carried by the allo-T cells.

Thus, these constructs will serve to elicit humoral and cellular immunity against HIV by piggybacking the HIV genome off the allogeneic T cells, rejection of which is certain when injected into a mismatched host.

The final allo-T cell product will be used as a protective vaccine against HIV. The cells would be injected several times, first shot would serve as a primer, and the following one or more shots as booster shots. It is expected that dosing options, and optimizing the safe dose/number of the injected cells, as well as the number injections and their timing will be determined by further safety/efficacy studies.

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein and unless otherwise indicated, the term “about” is intended to mean ±5% of the value it modifies. Thus, about 100 means 95 to 105. Additionally, the term “about” modifies a term in a series of terms, such as “about 1, 2, 3, 4, or 5” it should be understood that the term “about” modifies each of the members of the list, such that “about 1, 2, 3, 4, or 5” can be understood to mean “about 1, about 2, about 3, about 4, or about 5.” The same is true for a list that is modified by the term “at least” or other quantifying modifier, such as, but not limited to, “less than,” “greater than,” and the like.

As used herein and in the appended claims, the singular forms “a”, “an” and “the” include plural reference unless the context clearly dictates otherwise.

As used herein, the terms “comprise,” “have,” “has,” and “include” and their conjugates, as used herein, mean “including but not limited to.” While various compositions, and methods are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

Chemokine (C-X-C motif) ligand 13 (CXCL13), also known as B lymphocyte chemoattractant (BLC) or B cell-attracting chemokine 1 (BCA-1), is a protein ligand that in humans is encoded by the CXCL13 gene. CXCR5 is the receptor for CXCL13. Chemokines expression starts a positive loop of recruitment and stimulation of lymphocytes. Overexpressing CXCL13 in intestinal epithelial cells promoted a marked increase in the number of B cells in the lamina propria and an increase in the size and number of lymphoid follicles in the small intestine. These results suggest that overexpression of CXCL13 in the intestine during inflammatory conditions favors mobilization of B cells and of LTi and NK cells with immunomodulatory and reparative functions. In some embodiments CXCL13 is encoded by a nucleic acid molecule comprising the sequence of:

(SEQ ID NO: 1)
ATGAAGTTCATCTCGACATCTCTGCTTCTCATGCTGCTGGTCAGCAGCCT
CTCTCCAGTCCAAGGTGTTCTGGAGGTCTATTACACAAGCTTGAGGTGTA
GATGTGTCCAAGAGAGCTCAGTCTTTATCCCTAGACGCTTCATTGATCGA
ATTCAAATCTTGCCCCGTGGGAATGGTTGTCCAAGAAAAGAAATCATAGT
CTGGAAGAAGAACAAGTCAATTGTGTGTGTGGACCCTCAAGCTGAATGGA
TACAAAGAATGATGGAAGTATTGAGAAAAAGAAGTTCTTCAACTCTACCA
GTTCCAGTGTTTAAGAGAAAGATTCCC.

The Genbank accession number for the nucleic acid sequence is NM_006419.2, which is hereby incorporated by reference in its entirety. In some embodiments, the amino acid sequence of CXCL13 is

(SEQ ID NO: 3)
MKFISTSLLLMLLVSSLSPVQGVLEVYYTSLRCRCVQESSVFIPRRFIDR
IQILPRGNGCPRKEIIVWKKNKSIVCVDPQAEWIQRMMEVLRKRSSSTLP
VPVFKRKIP

Due to the degeneracy of the genetic code the sequence of SEQ ID NO: 1 is simply a non-limiting example and other nucleic acid sequences can be used to express CXCL13. In some embodiments, the nucleic acid sequence encoding SEQ ID NO: 3 is at least 85%, 90%, 91%, 92%, 93%, 94%, 95%, 96%, 97%, 98%, or 99% identical to SEQ ID NO: 1. In some embodiments, the protein of CXCL13 is at least 85%, 90%, 91%, 92%, 93%, 94%, 95%, 96%, 97%, 98%, or 99% identical to SEQ ID NO: 3. The sequence can contain conservative substitutions (mutations) that do not impact the activity of CXCL13.

CD40 ligand (CD40L), also called CD154, is a protein that is a member of the TNF superfamily of molecules. It binds to CD40 on antigen-presenting cells (APC), which leads to many effects depending on the target cell type. In total CD40L has three binding partners: CD40, α5β1 integrin and αIIbβ3. CD154 acts as a costimulatory molecule and is particularly important on a subset of T cells called T follicular helper cells (TFH cells). On TFH cells, CD40L promotes B cell maturation and function by engaging CD40 on the B cell surface and therefore facilitating cell-cell communication. CD40L stable expression allows the recombinant allogeneic CD4+ to produce IL-12 to overcome immunosuppression and to trigger memory T cell differentiation. In some embodiments CXCL13 is encoded by a nucleic acid molecule comprising the sequence of:

(SEQ ID NO: 2)
ATGATCGAAACATACAACCAAACTTCTCCCCGATCTGCGGCCACTGGACT
GCCCATCAGCATGAAAATTTTTATGTATTTACTTACTGTTTTTCTTATCA
CCCAGATGATTGGGTCAGCACTTTTTGCTGTGTATCTTCATAGAAGGTTG
GACAAGATAGAAGATGAAAGGAATCTTCATGAAGATTTTGTATTCATGAA
AACGATACAGAGATGCAACACAGGAGAAAGATCCTTATCCTTACTGAACT
GTGAGGAGATTAAAAGCCAGTTTGAAGGCTTTGTGAAGGATATAATGTTA
AACAAAGAGGAGACGAAGAAAGAAAACAGCTTTGAAATGCAAAAAGGTGA
TCAGAATCCTCAAATTGCGGCACATGTCATAAGTGAGGCCAGCAGTAAAA
CAACATCTGTGTTACAGTGGGCTGAAAAAGGATACTACACCATGAGCAAC
AACTTGGTAACCCTGGAAAATGGGAAACAGCTGACCGTTAAAAGACAAGG
ACTCTATTATATCTATGCCCAAGTCACCTTCTGTTCCAATCGGGAAGCTT
CGAGTCAAGCTCCATTTATAGCCAGCCTCTGCCTAAAGTCCCCCGGTAGA
TTCGAGAGAATCTTACTCAGAGCTGCAAATACCCACAGTTCCGCCAAACC
TTGCGGGCAACAATCCATTCACTTGGGAGGAGTATTTGAATTGCAACCAG
GTGCTTCGGTGTTTGTCAATGTGACTGATCCAAGCCAAGTGAGCCATGGC
ACTGGCTTCACGTCCTTTGGCTTACTCAAACTC

The Genbank accession number for the nucleic acid sequence is NM_000074.2, which is hereby incorporated by reference in its entirety. In some embodiments, the amino acid sequence of CD40L is

(SEQ ID NO: 4)
MIETYNQTSPRSAATGLPISMKIFMYLLTVFLITQMIGSALFAVYLHRRL
DKIEDERNLHEDFVFMKTIQRCNTGERSLSLLNCEEIKSQFEGFVKDIML
NKEETKKENSFEMQKGDQNPQIAAHVISEASSKTTSVLQWAEKGYYTMSN
NLVTLENGKQLTVKRQGLYYIYAQVTFCSNREASSQAPFIASLCLKSPGR
EERILLRAANTHSSAKPCGQQSIHLGGVFELQPGASVEVNVTDPSQVSHG
TGETSFGLLKL

Due to the degeneracy of the genetic code the sequence of SEQ ID NO: 2 is simply a non-limiting example and other nucleic acid sequences can be used to express CD40L. In some embodiments, the nucleic acid sequence encoding SEQ ID NO: 4 is at least 85%, 90%, 91%, 92%, 93%, 94%, 95%, 96%, 97%, 98%, or 99% identical to SEQ ID NO: 2. In some embodiments, the protein of CD40L is at least 85%, 90%, 91%, 92%, 93%, 94%, 95%, 96%, 97%, 98%, or 99% identical to SEQ ID NO: 4. The sequence can contain conservative substitutions (mutations) that do not impact the activity of CD40L.

As used herein, the term “heterologous” when referencing to a nucleic acid molecule in a cell means that the nucleic acid molecule is not native to the genome of the naturally occurring cell even if the cell has a similar sequence or genetic sequence that encodes for the same protein or sequence. For example, a cell that comprises a heterologous nucleic acid molecule encoding CD40L and/or CXCL13 refers to a cell that has been modified to contain the a nucleic acid molecule(s) that encodes CD40L and/or CXCL13. This can be done by transfection or transduction or other genome editing techniques, such as, but not limited to, CRISPR and the like. When the term “heterologous” is used in reference to a protein in a cell it refers to a protein that is encoded for by a heterologous nucleic acid molecule. Thus a cell can contain the same protein that is native to the cell, which is a protein not encoded for by a heterologous nucleic acid molecule, and that is heterologous to the cell, which is a protein that is encoded for by a heterologous nucleic acid molecule. The heterologous nucleic acid molecule can be transiently in the cell or stably present in the cell either by being inserted into the cell's genome or by the presence of an episomal plasmid in the cell. The heterologous nucleic acid molecule can also be introduced into the cell through viral transduction, which can also be stably integrated into the genome of the cell.

The recombinant cells expressing heterologous proteins can also be modified to express a target antigen to which an immune response is desired to be generated against. For example, the target antigen can be a HIV protein, or antigenic fragment thereof. In some embodiments, the cell, such as a T-cell, or CD4+ T-cell, heterologously expressing CD40L and/or CXCL13 also heterologously expresses a target antigen. In some embodiments, the additional nucleic acid molecule comprises a human immunodeficiency virus (HIV) genome. In some embodiments, the HIV genomic nucleic acid comprises a mutation in the retroviral reverse transcriptase, and further wherein the HIV genomic nucleic acid does not encode a retroviral packaging signal, creating a disabled HIV genomic construct. In some embodiments, the target antigen is one or more of HIV Tat (full length or isoforms of 72 and 101 amino acids in length), Rev, Pol, GP120, GP160, GP41, env, Gag, Gag-Pol, Nef, Vpr, Vpu, Vif, and the like, or any combination thereof. In some embodiments, the target antigen is any HIV protein. In some embodiments, the cells heterologously expressing CD40L and/or CXCL13 express each of the HIV proteins. In some embodiments, the cells heterologously express at least, or exactly, 1, 2, 3, 4, 5, 6, 7, 8, 9, or 10 HIV proteins, or fragments thereof. In some embodiments, the target antigen is HIV Tat (full length or isoforms of 72 and 101 amino acids in length). In some embodiments, the target antigen is HIV Rev. In some embodiments, the target antigen is HIV Pol. In some embodiments, the target antigen is HIV GP120. In some embodiments, the target antigen is HIV GP160. In some embodiments, the target antigen is HIV GP41. In some embodiments, the target antigen is HIV env. In some embodiments, the target antigen is HIV Gag. In some embodiments, the target antigen is HIV Gag-Pol. In some embodiments, the target antigen is HIV Nef. In some embodiments, the target antigen is HIV Vpr. In some embodiments, the target antigen is HIV Vpu. In some embodiments, the target antigen is HIV Vif. Any of these antigens can be heterologously expressed in the cell heterologously expressing CD40L and/or CXCL13. In some embodiments, instead of HIV antigens, SHIV antigens are used and the equivalent antigens can be used in place of the antigens provided for herein.

If a HIV genome is used, HIV genomic plasmids are commercially available and exemplary mutations and systems are described for example in Mol Ther. 2017 August 2;25(8):1790-1804. Similarly, commercially available plasmid backbone pVAX1, carrying Chinese HIV-1 subtype C/B=env and gag genes in one plasmid and pol and a nef/tat construct designed to express a fusion protein in the second plasmid, with plasmids mixed in a 1:1 ratio, is also available (See Advax, San Diego, Calif. and Clinical and Vaccine Immunology, Volume 20 Number 3, March 2013, p. 397-408.) This is a non-limiting example and other constructs can be used.

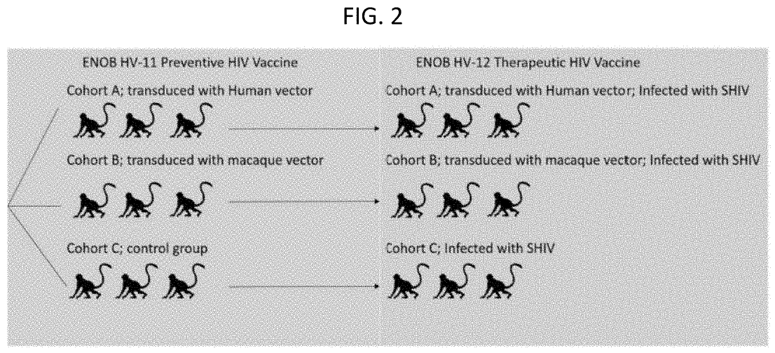

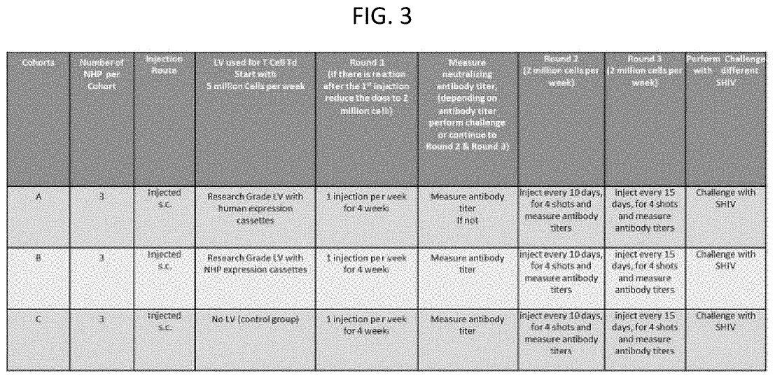

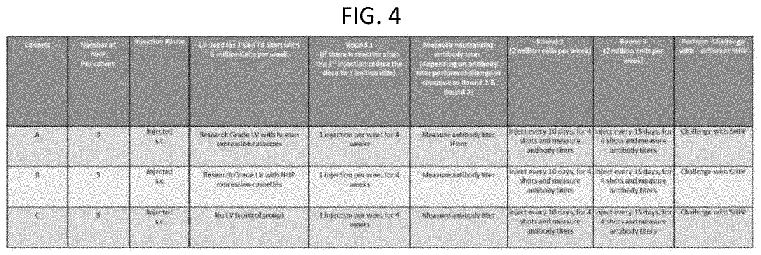

Vector maps of the exemplary constructs used to make the recombinant allogeneic CD4+ cells are shown in FIG. 1 and FIG. 1B, and outlines and schematics for testing these constructs are shown in FIG. 2, FIG. 3, and FIG. 4.

The terms “co-administration” or the like, are meant to encompass administration of the selected therapeutic agents to a single patient, and are intended to include treatment regimens in which the agents are administered by the same or different route of administration or at the same or different time.

As used herein, the term “agonist” refers to a compound, the presence of which results in a biological activity of a protein that is the same as the biological activity resulting from the presence of a naturally occurring ligand for the protein.

As used herein, the term “partial agonist” refers to a compound the presence of which results in a biological activity of a protein that is of the same type as that resulting from the presence of a naturally occurring ligand for the protein, but of a lower magnitude.

As used herein, the term “antagonist” refers to a compound, the presence of which results in a decrease in the magnitude of a biological activity of a protein. In certain embodiments, the presence of an antagonist results in complete inhibition of a biological activity of a protein. In certain embodiments, an antagonist is an inhibitor.

“Administering” when used in conjunction with a therapeutic composition (e.g. allogeneic T-cell-based protective HIV vaccine, recombinant allogeneic CD4+ based vaccine and compositions comprising these products) means to administer a therapeutic directly into or onto a target tissue or to administer a therapeutic to a patient whereby the therapeutic positively impacts the tissue to which it is targeted.

The term “subject” or “patient” as used herein includes, but is not limited to, humans and non-human vertebrates such as wild, domestic, and farm animals. In some embodiments, the subject or patient, which can be used interchangeably, can be a non-human primate. In certain embodiments, the subject or patient described herein is an animal. In certain embodiments, the subject or patient is a mammal. In certain embodiments, the subject is a human. In certain embodiments, the subject or patient is a non-human animal. In certain embodiments, the subject or patient is a non-human mammal. In certain embodiments, the subject or patient is a domesticated animal, such as a dog, cat, cow, pig, horse, sheep, or goat. In certain embodiments, the subject or patient is a companion animal such as a dog or cat. In certain embodiments, the subject or patient is a livestock animal such as a cow, pig, horse, sheep, or goat. In certain embodiments, the subject or patient is a zoo animal. In another embodiment, the subject or patient is a research animal such as a rodent, dog, or non-human primate. In certain embodiments, the subject or patient is a non-human transgenic animal such as a transgenic mouse or transgenic pig.

The term “inhibit” includes the administration of a therapeutic of embodiments herein to prevent the onset of the symptoms, alleviating the symptoms, or eliminating the disease, condition or disorder.

By “pharmaceutically acceptable”, it is meant the carrier, diluent or excipient must be compatible with the other ingredients of the therapeutic and not deleterious to the recipient thereof.

The terms “treat,” “treated,” or “treating” as used herein refers to both therapeutic treatment and prophylactic or preventative measures, wherein the object is to inhibit, prevent or slow down (lessen) an undesired physiological condition, disorder or disease, or to improve, inhibit, or otherwise obtain beneficial or desired clinical results. For the purposes of this invention, beneficial or desired clinical results include, but are not limited to, improvement or alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. The antibodies may exist in a variety of forms including, for example, polyclonal antibodies, monoclonal antibodies, Fv, Fab and F(ab)2, as well as single chain antibodies and humanized antibodies.

The term “antibody fragment” refers to a portion of an intact antibody and refers to the antigenic determining variable regions of an intact antibody. Examples of antibody fragments include, but are not limited to, Fab, Fab', F(ab')2, and Fv fragments, linear antibodies, scFv antibodies, and multispecific antibodies formed from antibody fragments.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the embodiments include, but are not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tissue sample suspected of containing a virus, a cell or a biological fluid.

The term “auto-antigen” means any self-antigen which is mistakenly recognized by the immune system as being foreign. Auto-antigens comprise, but are not limited to, cellular proteins, phosphoproteins, cellular surface proteins, cellular lipids, nucleic acids, glycoproteins, including cell surface receptors.

As used herein, the term “autologous” is meant to refer to any material derived from the same individual to which it is later to be re-introduced into the individual.

The term “allogeneic” as used herein, refers to HLA or MHC loci that are antigenically distinct. Thus, cells or tissue transferred from the same species can be antigenically distinct. Syngeneic mice can differ at one or more loci (congenics) and allogeneic mice can have the same background.

The term “antigen” as used herein is defined as a molecule that provokes an immune response. This immune response may involve either antibody production, or the activation of specific immunologically-competent cells, or both.

“Xenogeneic” refers to a graft derived from an animal of a different species.

The term “donor” refers to a mammal, for example, a human, that is not the patient recipient. The donor may, for example, have HLA identity with the recipient, or may have partial or greater HLA disparity with the recipient.

The term “haploidentical” as used with reference to cells, cell types and/or cell lineages, herein refers to cells sharing a haplotype or cells having substantially the same alleles at a set of closely linked genes on one chromosome. A haploidentical donor does not have complete HLA identity with the recipient, there is a partial HLA disparity.

T cells and Activated T cells (including CD3+ cells): T cells (also referred to as T lymphocytes) belong to a group of white blood cells referred to as lymphocytes. Lymphocytes generally are involved in cell-mediated immunity. The “T” in “T cells” refers to cells derived from or whose maturation is influence by the thymus. T cells can be distinguished from other lymphocytes types such as B cells and Natural Killer (NK) cells by the presence of cell surface proteins known as T cell receptors. The term “activated T cells” as used herein, refers to T cells that have been stimulated to produce an immune response (e.g., clonal expansion of activated T cells) by recognition of an antigenic determinant presented in the context of a Class II major histocompatibility (MHC) marker. T-cells are activated by the presence of an antigenic determinant, cytokines and/or lymphokines and cluster of differentiation cell surface proteins (e.g., CD3, CD4, CD8, the like and combinations thereof). Cells that express a cluster of differential protein often are said to be “positive” for expression of that protein on the surface of T-cells (e.g., cells positive for CD3 or CD 4 expression are referred to as CD3+ or CD4+). CD3 and CD4 proteins are cell surface receptors or co-receptors that may be directly and/or indirectly involved in signal transduction in T cells.

The term “peripheral blood” as used herein, refers to cellular components of blood (e.g., red blood cells, white blood cells and platelets), which are obtained or prepared from the circulating pool of blood and not sequestered within the lymphatic system, spleen, liver or bone marrow.

“Peripheral blood mononuclear cells”, “PBMCs”, or “mononuclear cells” refer to mononuclear cells separated from peripheral blood typically used for anti-cancer immunotherapy. The peripheral blood mononuclear cells can be obtained from human blood collected using known methods such as the Ficoll-Hypaque density gradient method.

According to one exemplary embodiment of the present invention, “peripheral blood mononuclear cells” may be obtained from any suitable person. The source of the donor lymphocytic cells, including sources such as peripheral blood mononuclear cells, as used herein may be allogeneic or autologous to the recipient patient for isolation of the desired lymphocytic cells including: CD4+ cells , NK cells, γδT cells, iNKT cells, CD3 T cells, or other combinations for use in the anti-HIV, protective and/or or therapeutic methods according to the present invention. In certain embodiments, the recombinant cells are allogeneic, and in others, they may be autologous.

As used herein, the term “ex vivo” refers to “outside” the body.

A “disease” is a state of health of a subject wherein the subject cannot maintain homeostasis, and wherein if the disease is not ameliorated then the animal's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system. A heterologous nucleic acid molecule in a cell is an exogenous nucleic acid molecule.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. An “isolated” biological component (such as a nucleic acid, protein or cell) has been substantially separated or purified away from other biological components (such as cell debris, other proteins, nucleic acids or cell types). Biological components that have been “isolated” include those components purified by standard purification methods.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

As used herein, recombinant generally refers to the following: A recombinant nucleic acid or protein is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, for example, by genetic engineering techniques.

As used herein, the following abbreviations for the commonly occurring nucleic acid bases are used. “A” refers to adenosine, “C” refers to cytosine, “G” refers to guanosine, “T” refers to thymidine, and “U” refers to uridine.

The term “leukocytes” or “white blood cell” as used herein refers to any immune cell, including monocytes, neutrophils, eosinophils, basophils, and lymphocytes. The term “lymphocytes” as used herein refer to cells commonly found in lymph, and include natural killer cells (NK cells), T-cells, and B-cells. It will be appreciated by one of skill in the art that the above listed immune cell types can be divided into further subsets.

The term “tumor infiltrating leukocytes” as used herein refers to leukocytes that are present in a solid tumor.

The term “blood sample” as used herein refers to any sample prepared from blood, such as plasma, blood cells isolated from blood, and so forth.

The term “purified sample” as used herein refers to any sample in which one or more cell subsets are enriched. A sample may be purified by the removal or isolation of cells based on characteristics such as size, protein expression, and so forth.

The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compositions, and additional pharmaceutical agents.

The compositions and cells provided herein can be administered by any suitable methods. The compositions and cells of the embodiments provided for herein may be in a variety of forms. These include, for example, liquid and semi-solid, such as liquid solutions (e.g., injectable and infusible solutions), dispersions or suspensions, liposomes and suppositories. The form depends on the intended mode of administration and therapeutic application. In some embodiments, compositions are in the form of injectable or infusible solutions. In some embodiments, the mode of administration is parenteral (e.g., intravenous, subcutaneous, intraperitoneal, intramuscular). In some embodiments, the therapeutic composition (pharmaceutical composition) is administered by intravenous infusion or injection. In some embodiments, the therapeutic molecule is administered by intramuscular or subcutaneous injection. In some embodiments, the therapeutic composition is administered locally, e.g., by injection to a target site. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

In general, the nature of a suitable carrier or vehicle for delivery will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

In some embodiments, compositions, whether they be solutions, suspensions or other like form, may include one or more of the following: DMSO, sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

Diseases that the compositions and methods described herein can treat include microbial infections such as a viral infection.

As used herein, “viral infection” is meant as an infection caused by the presence of a virus in the body. Viral infections include chronic or persistent viral infections, which are viral infections that are able to infect a host and reproduce within the cells of a host over a prolonged period of time-usually weeks, months or years, before proving fatal. Viruses giving rise to chronic infections include, for example, the human papilloma viruses (HPV), Herpes simplex, and other herpes viruses, the viruses of hepatitis B and C as well as other hepatitis viruses, human immunodeficiency virus, and the measles virus, all of which can produce important clinical diseases. Prolonged infection may ultimately lead to the induction of disease which may be, e.g., in the case of hepatitis C virus liver cancer, fatal to the patient. Other chronic viral infections which may be treated in accordance with the present invention include Epstein Barr virus (EBV), as well as other viruses such as those which may be associated with tumors.

Examples of viral infections which can be treated or prevented with the recombinant allogeneic CD4+ HIV vaccine cells or composition comprising such cells and methods described herein include, but are limited to, viral infections caused by retroviruses (e.g., human T-cell lymphotrophic virus (HTLV), particularly types I and II and human immunodeficiency virus (HIV)). The treatment and/or prevention of a viral infection includes, but is not limited to, alleviating one or more symptoms associated with said infection, the inhibition, reduction or suppression of viral replication and/or viral load, and/or the enhancement of the immune response.

As used herein, “immunodeficient” means lacking in at least one essential function of the immune system. As used herein, an “immunodeficient subject” (such as a human) is one lacking specific components of the immune system or lacking function of specific components of the immune system (such as, for example, B cells, T cells, NK cells or macrophages). In some cases, an immunodeficient subject comprises one or more genetic alterations that prevent or inhibit the development of functional immune cells (such as B cells, T cells or NK cells). In some examples, the genetic alteration is in IL17 or IL17 receptor.

As used herein, “immunosuppressed” refers to a reduced activity or function of the immune system. A subject can be immunosuppressed, for example, due to treatment with an immunosuppressant compound or as a result of a disease or disorder (for example, immunosuppression that results from HIV infection or due to a genetic defect). In some cases, immunosuppression occurs as the result of a genetic mutation that prevents or inhibits the development of functional immune cells, such as T cells.

In some embodiments of the invention, a “therapeutically effective amount” is an amount of recombinant allogeneic or autologous T cell, such as a CD4+, with a target antigen, such as a HIV protein, vaccine cells or composition comprising such cells, as described herein that results in a reduction in viral titer by at least 2.5%, at least 5%, at least 10%, at least 15%, at least 25%, at least 35%, at least 45%, at least 50%, at least 75%, at least 85%, by at least 90%, at least 95%, or at least 99% in a subject/patient/animal administered the recombinant allogeneic CD4+ HIV vaccine cells or composition comprising such cells and treated with a related method described herein, relative to the viral titer or microbial titer in an animal or group of animals (e.g., two, three, five, ten or more animals) not administered a recombinant T-cell (e.g. CD4+ T-cell) vaccine cells, or composition comprising such cells of the invention.

In certain embodiments, the recombinant allogeneic or autologous CD4+ HIV vaccine cells or compositions comprising such cells can be administered simultaneously with anti-microbial, anti-viral and/or other therapeutic agents. Alternatively, recombinant allogeneic or autologous CD4+ HIV vaccine cells or composition comprising such cells can be administered at selected times in advance of times when anti-microbial, anti-viral and other therapeutic agents are administered.

As provided herein, the recombinant T-cells (e.g. CD4+ cells) can, in some embodiments be allogeneic as compared to the patient being administered the cells or they can be autologous, or HLA matched.

Cell Sources

Peripheral blood mononuclear cells (PBMCs) can be isolated by Ficoll-Hypaque density gradient centrifugation of samples obtained from discarded, de-identified leukocyte reduction filters (American Red Cross), or blood donations from healthy volunteers with informed consent. Descriptions of cell populations, sources and methods for selecting or enriching for desired cell types can be found, for example in: U.S. Pat. No. 9,347,044. Populations of cells for use in the methods described herein for treating mammals must be species matched, such as human cells. The cells may be obtained from an animal, e.g., a human patient. If the cells are obtained from an animal, they may have been established in culture first, e.g., by transformation; or more preferably, they may have been subjected to preliminary purification methods. For example, a cell population may be manipulated by positive or negative selection based on expression of cell surface markers; stimulated with one or more antigens in vitro or in vivo; treated with one or more biological modifiers in vitro or in vivo; or a combination of any or all of these. In an illustrative embodiment, a cell population is subjected to negative selection for depletion of non-T cells and/or particular T cell subsets. Negative selection can be performed on the basis of cell surface expression of a variety of molecules, including B cell markers such as CD19, and CD20; monocyte marker CD14; the NK cell marker CD56. Alternately, a cell population may be subjected to negative selection for depletion of non-CD34.sup.+hematopoietic cells and/or particular hematopoietic cell subsets. Negative selection can be performed on the basis of cell surface expression of a variety of molecules, such as a cocktail of antibodies (e.g., CD2, CD3, CD11b, CD14, CD15, CD16, CD19, CD56, CD123, and CD235a) which may be used for separation of other cell types, e.g., via MACS or column separation.

Populations of cells include peripheral blood mononuclear cells (PBMC), whole blood or fractions thereof containing mixed populations, spleen cells, bone marrow cells, tumor infiltrating lymphocytes, cells obtained by leukapheresis, biopsy tissue, lymph nodes, e.g., lymph nodes draining from a tumor. Suitable donors include immunized donors, non-immunized (naive) donors, treated or untreated donors. A “treated” donor is one that has been exposed to one or more biological modifiers. An “untreated” donor has not been exposed to one or more biological modifiers.

Methods of obtaining populations of cells comprising a T cell are well known in the art. For example, peripheral blood mononuclear cells (PBMC) can be obtained as described according to methods known in the art. Examples of such methods are set forth in the Examples and is discussed by Kim et al. (1992); Biswas et al. (1990); Biswas et al. (1991).

It is also possible to obtain a cell sample from a subject, and then to enrich it for a desired cell type. For example, PBMCs can be isolated from blood as described herein. Counter-flow centrifugation (elutriation) can be used to enrich for T cells from PBMCs. Cells can also be isolated from other cells using a variety of techniques, such as isolation and/or activation with an antibody binding to an epitope on the cell surface of the desired cell type, for example, some T-cell isolation kits use antibody conjugated beads to both activate the cells and then allow column separation with the same beads. Another method that can be used includes negative selection using antibodies to cell surface markers to selectively enrich for a specific cell type without activating the cell by receptor engagement.

Bone marrow cells may be obtained from iliac crest, femora, tibiae, spine, rib or other medullary spaces. Bone marrow may be taken out of the patient and isolated through various separations and washing procedures. A known procedure for isolation of bone marrow cells comprises the following steps: a) centrifugal separation of bone marrow suspension in three fractions and collecting the intermediate fraction, or buffy coat; b) the buffy coat fraction from step (a) is centrifuged one more time in a separation fluid, commonly Ficoll (a trademark of Pharmacia Fine Chemicals AB), and an intermediate fraction which contains the bone marrow cells is collected; and c) washing of the collected fraction from step (b) for recovery of re-transfusable bone marrow cells.

If one desires to use a population of cells enriched in T cells, such populations of cells can be obtained from a mixed population of cells by leukapheresis and mechanical apheresis using a continuous flow cell separator. For example, T cells can be isolated from the buffy coat by any known method, including separation over Ficoll-Hypaque™ gradient, separation over a Percoll gradient, or elutriation.

### Methods of Viral Vector-Mediated Transfer

In certain embodiments, a transgene is incorporated into a viral particle to mediate gene transfer to a cell. Typically, the virus simply will be exposed to the appropriate host cell under physiologic conditions, permitting uptake of the virus. (See, U.S. Pat. No. 9,089,520) The present methods are advantageously employed using a variety of viral vectors, as discussed below, and also including lentiviral vectors.

1. Adenovirus

Adenovirus is particularly suitable for use as a gene transfer vector because of its mid-sized DNA genome, ease of manipulation, high titer, wide target-cell range, and high infectivity. The roughly 36 kb viral genome is bounded by 100-200 base pair (bp) inverted terminal repeats (ITR), in which are contained cis-acting elements necessary for viral DNA replication and packaging. The early (E) and late (L) regions of the genome that contain different transcription units are divided by the onset of viral DNA replication.

The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan, M. J. (1990) Radiother Oncol., 19, 197-218). The products of the late genes (L1, L2, L3, L4 and L5), including the majority of the viral capsid proteins, are expressed only after significant processing of a single primary transcript issued by the major late promoter (MLP). The MLP (located at 16.8 map units) is particularly efficient during the late phase of infection, and all the mRNAs issued from this promoter possess a 5′ tripartite leader (TL) sequence, which makes them useful for translation.

In order for adenovirus to be optimized for gene therapy, it is necessary to maximize the carrying capacity so that large segments of DNA can be included. It also is very desirable to reduce the toxicity and immunologic reaction associated with certain adenoviral products. The two goals are, to an extent, coterminous in that elimination of adenoviral genes serves both ends. By practice of the present methods, it is possible to achieve both these goals while retaining the ability to manipulate the therapeutic constructs with relative ease.

The large displacement of DNA is possible because the cis elements required for viral DNA replication all are localized in the inverted terminal repeats (ITR) (100-200 bp) at either end of the linear viral genome. Plasmids containing ITR's can replicate in the presence of a non-defective adenovirus (Hay, R. T., et al., J Mol. Biol. 1984 Jun. 5; 175(4):493-510). Therefore, inclusion of these elements in an adenoviral vector may permits replication.

In addition, the packaging signal for viral encapsulation is localized between 194-385 bp (0.5-1.1 map units) at the left end of the viral genome (Hearing et al., J. (1987) Virol., 67, 2555-2558). This signal mimics the protein recognition site in bacteriophage lambda DNA where a specific sequence close to the left end, but outside the cohesive end sequence, mediates the binding to proteins that are required for insertion of the DNA into the head structure. E1 substitution vectors of Ad have demonstrated that a 450 bp (0-1.25 map units) fragment at the left end of the viral genome could direct packaging in 293 cells (Levrero et al., Gene, 101:195-202, 1991).

Previously, it has been shown that certain regions of the adenoviral genome can be incorporated into the genome of mammalian cells and the genes encoded thereby expressed. These cell lines are capable of supporting the replication of an adenoviral vector that is deficient in the adenoviral function encoded by the cell line. There also have been reports of complementation of replication deficient adenoviral vectors by “helping” vectors, e.g., wild-type virus or conditionally defective mutants.

Replication-deficient adenoviral vectors can be complemented, in trans, by helper virus. This observation alone does not permit isolation of the replication-deficient vectors, however, since the presence of helper virus, needed to provide replicative functions, would contaminate any preparation. Thus, an additional element was needed that would add specificity to the replication and/or packaging of the replication-deficient vector. That element derives from the packaging function of adenovirus.

It has been shown that a packaging signal for adenovirus exists in the left end of the conventional adenovirus map (Tibbetts et. al. (1977) Cell, 12, 243-249). Later studies showed that a mutant with a deletion in the E1A (194-358 bp) region of the genome grew poorly even in a cell line that complemented the early (E1A) function (Hearing and Shenk, (1983) J. Mol. Biol. 167, 809-822). When a compensating adenoviral DNA (0-353 bp) was recombined into the right end of the mutant, the virus was packaged normally. Further mutational analysis identified a short, repeated, position-dependent element in the left end of the Ad5 genome. One copy of the repeat was found to be sufficient for efficient packaging if present at either end of the genome, but not when moved toward the interior of the Ad5 DNA molecule (Hearing et al., J. (1987) Virol., 67, 2555-2558).

By using mutated versions of the packaging signal, it is possible to create helper viruses that are packaged with varying efficiencies. Typically, the mutations are point mutations or deletions. When helper viruses with low efficiency packaging are grown in helper cells, the virus is packaged, albeit at reduced rates compared to wild-type virus, thereby permitting propagation of the helper. When these helper viruses are grown in cells along with virus that contains wild-type packaging signals, however, the wild-type packaging signals are recognized preferentially over the mutated versions. Given a limiting amount of packaging factor, the virus containing the wild-type signals is packaged selectively when compared to the helpers. If the preference is great enough, stocks approaching homogeneity may be achieved.

To improve the tropism of ADV constructs for particular tissues or species, the receptor-binding fiber sequences can often be substituted between adenoviral isolates. For example the Coxsackie-adenovirus receptor (CAR) ligand found in adenovirus 5 can be substituted for the CD46-binding fiber sequence from adenovirus 35, making a virus with greatly improved binding affinity for human hematopoietic cells. The resulting “pseudotyped” virus, Ad5f35, has been the basis for several clinically developed viral isolates. Moreover, various biochemical methods exist to modify the fiber to allow re-targeting of the virus to target cells. Methods include use of bifunctional antibodies (with one end binding the CAR ligand and one end binding the target sequence), and metabolic biotinylation of the fiber to permit association with customized avidin-based chimeric ligands. Alternatively, one could attach ligands (e.g. anti-CD205 by heterobifunctional linkers (e.g. PEG-containing), to the adenovirus particle.

2. Retrovirus

The retroviruses are a group of single-stranded RNA viruses characterized by an ability to convert their RNA to double-stranded DNA in infected cells by a process of reverse-transcription (Coffin, (1990) In: Virology, ed., New York: Raven Press, pp. 1437-1500). The resulting DNA then stably integrates into cellular chromosomes as a provirus and directs synthesis of viral proteins. The integration results in the retention of the viral gene sequences in the recipient cell and its descendants. The retroviral genome contains three genes--gag, pol and env--that code for capsid proteins, polymerase enzyme, and envelope components, respectively. A sequence found upstream from the gag gene, termed psi, functions as a signal for packaging of the genome into virions. Two long terminal repeat (LTR) sequences are present at the 5′ and 3′ ends of the viral genome. These contain strong promoter and enhancer sequences and also are required for integration in the host cell genome (Coffin, 1990).

In order to construct a retroviral vector, a nucleic acid encoding a promoter is inserted into the viral genome in the place of certain viral sequences to produce a virus that is replication-defective. In order to produce virions, a packaging cell line containing the gag, pol and env genes but without the LTR and psi components is constructed (Mann et al., (1983) Cell, 33, 153-159). When a recombinant plasmid containing a human cDNA, together with the retroviral LTR and psi sequences is introduced into this cell line (by calcium phosphate precipitation for example), the psi sequence allows the RNA transcript of the recombinant plasmid to be packaged into viral particles, which are then secreted into the culture media (Nicolas, J. F., and Rubenstein, J. L. R., (1988) In: Vectors: a Survey of Molecular Cloning Vectors and Their Uses, Rodriquez and Denhardt, Eds.). Nicolas and Rubenstein; Temin et al., (1986) In: Gene Transfer, Kucherlapati (ed.), New York: Plenum Press, pp. 149-188; Mann et al., 1983). The media containing the recombinant retroviruses is collected, optionally concentrated, and used for gene transfer. Retroviral vectors are able to infect a broad variety of cell types. However, integration and stable expression of many types of retroviruses require the division of host cells (Paskind et al., (1975) Virology, 67, 242-248). An approach designed to allow specific targeting of retrovirus vectors recently was developed based on the chemical modification of a retrovirus by the chemical addition of galactose residues to the viral envelope. This modification could permit the specific infection of cells such as hepatocytes via asialoglycoprotein receptors, may this be desired.

A different approach to targeting of recombinant retroviruses was designed in which biotinylated antibodies against a retroviral envelope protein and against a specific cell receptor were used. The antibodies were coupled via the biotin components by using streptavidin (Roux et al., (1989) Proc. Nat'l Acad. Sci. USA, 86, 9079-9083). Using antibodies against major histocompatibility complex class I and class II antigens, the infection of a variety of human cells that bore those surface antigens was demonstrated with an ecotropic virus in vitro (Roux et al., 1989).

3. Adeno-Associated Virus

AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription.

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low-level expression of AAV rep proteins is believed to hold AAV structural expression in check, and helper virus infection is thought to remove this block.

The terminal repeats of the AAV vector can be obtained by restriction endonuclease digestion of AAV or a plasmid such as p201, which contains a modified AAV genome (Samulski et al., J. Virol., 61:3096-3101 (1987)), or by other methods, including but not limited to chemical or enzymatic synthesis of the terminal repeats based upon the published sequence of AAV. It can be determined, for example, by deletion analysis, the minimum sequence or part of the AAV ITRs which is required to allow function, i.e., stable and site-specific integration. It can also be determined which minor modifications of the sequence can be tolerated while maintaining the ability of the terminal repeats to direct stable, site-specific integration.

AAV-based vectors have proven to be safe and effective vehicles for gene delivery in vitro, and these vectors are being developed and tested in pre-clinical and clinical stages for a wide range of applications in potential gene therapy, both ex vivo and in vivo (Carter and Flotte, (1995) Ann. N.Y. Acad. Sci., 770; 79-90; Chatteijee, et al., (1995) Ann. N.Y. Acad. Sci., 770, 79-90; Ferrari et al., (1996) J. Virol., 70, 3227-3234; Fisher et al., (1996) J. Virol., 70, 520-532; Flotte et al., Proc. Nat'l Acad. Sci. USA, 90, 10613-10617, (1993); Goodman et al. (1994), Blood, 84, 1492-1500; Kaplitt et al., (1994) Nat'l Genet., 8, 148-153; Kaplitt, M. G., et al., Ann Thorac Surg. 1996 December; 62(6):1669-76; Kessler et al., (1996) Proc. Nat'l Acad. Sci. USA, 93, 14082-14087; Koeberl et al., (1997) Proc. Nat'l Acad. Sci. USA, 94, 1426-1431; Mizukami et al., (1996) Virology, 217, 124-130).

AAV-mediated efficient gene transfer and expression in the lung has led to clinical trials for the treatment of cystic fibrosis (Carter and Flotte, 1995; Flotte et al., Proc. Nat'l Acad. Sci. USA, 90, 10613-10617, (1993)). Similarly, the prospects for treatment of muscular dystrophy by AAV-mediated gene delivery of the dystrophin gene to skeletal muscle, of Parkinson's disease by tyrosine hydroxylase gene delivery to the brain, of hemophilia B by Factor IX gene delivery to the liver, and potentially of myocardial infarction by vascular endothelial growth factor gene to the heart, appear promising since AAV-mediated transgene expression in these organs has recently been shown to be highly efficient (Fisher et al., (1996) J. Virol., 70, 520-532; Flotte et al., 1993; Kaplitt et al., 1994; 1996; Koeberl et al., 1997; McCown et al., (1996) Brain Res., 713, 99-107; Ping et al., (1996) Microcirculation, 3, 225-228; Xiao et al., (1996) J. Virol., 70, 8098-8108).

4. Lentiviral Vectors

In certain embodiments, the CXCL13 and CD40L are transduced into the CD4 T cells, T cell subsets and/or T cell progenitors with lentiviruses, gamma-retroviruses, alpha-retroviruses or adenoviruses, by electroporation, or by transfection of nucleic acids, proteins, site-specific nucleases, self-replicating RNA viruses or integration-deficient lentiviral vectors. (for such vectors see, U.S. Pat. No. 10,131,876).

In certain embodiments, the recombinant modification of CD4 T cells, T cell subsets and/or T cell progenitors may be performed by transduction, transfection or electroporation.

Preferably, transduction is performed with lentiviruses, gamma-, alpha-retroviruses or adenoviruses or with electroporation or transfection by nucleic acids (DNA, mRNA, miRNA, antagomirs, ODNs), proteins, site-specific nucleases (zinc finger nucleases, TALENs, CRISP/R), self-replicating RNA viruses (e.g. equine encephalopathy virus) or integration-deficient lentiviral vectors.

More preferentially, recombinant modification of CD4 T cells, T cell subsets and/or T cell progenitors may be performed by transducing said cells with lentiviral vectors (See, Cockrell Adam S et al., “Gene delivery by lentivirus vectors”, Molecular Biotechnology, vol. 36, No. 3, Jul. 2007.)

Lentiviral vectors with the VSVG pseudotype enable efficient transduction under automated manufacturing method. However, the present methods are entirely suitable for the use of any type of lentiviral vector (with e.g. measles virus (ML-LV), gibbon ape leukaemia virus (GALV), feline endogenous retrovirus (RD114), baboon endogenous retrovirus (BaEV) derived pseudotyped envelopes). Other viral vectors such as gamma or alpha retroviral vectors can be used. Transduction enhancer reagents can be added when necessary using the automated manufacturing described in this invention.

5. Other Viral Vectors

Other viral vectors can be employed as expression constructs in the present methods and compositions. Vectors derived from viruses such as vaccinia virus (Ridgeway, (1988) In: Vectors: A survey of molecular cloning vectors and their uses, pp. 467-492; Baichwal and Sugden, (1986) In, Gene Transfer, pp. 117-148; Coupar et al., Gene, 68:1-10, 1988) canary poxvirus, and herpes viruses are employed. These viruses offer several features for use in gene transfer into various mammalian cells.

Once the construct has been delivered into the cell, the nucleic acid encoding the transgene are positioned and expressed at different sites. In certain embodiments, the nucleic acid encoding the transgene is stably integrated into the genome of the cell. This integration is in the cognate location and orientation via homologous recombination (gene replacement) or it is integrated in a random, non-specific location (gene augmentation). In yet further embodiments, the nucleic acid is stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

These methods of introducing heterologous nucleic acid molecules into a cell are non-liiting and any method can be used. These can be used to heterologously express CD40L, CXCL13, and/or the target antigen, which can be a HIV protein as provided for herein.

### Methods for Treating a Disease

The present methods also encompass methods of treatment or prevention of a disease where administration of cells by, for example, infusion, may be beneficial. In some embodiments, the disease is a viral infection. In some embodiments, the infection is HIV infection. In some embodiments, the methods comprise administering a composition or cell as provided for herein to the subject with the viral infeciton. In some embodiments, the method increases an immune response, such as humoral and/or cellular immune response. In some embodiments, the immune response is against a HIV infection.

In some embodiments, methods of treating HIV in a subject are provided. In some embodiments, the methods comprise administering to the subject a composition comprising administering an effective amount of any of the cells provided herein. In some embodiments, the composition can be referred to a pharmaceutical composition. As provided herein, the composition can be administered by any suitable route. In some embodiments, the composition is administered intravenously or by infusion. In some embodiments, cells are allogeneic to, or not HLA-matched to the subject. In some embodiments, the cells are autologous to the subject. In some embodiments, the dosage of cells in the composition is from about 1×10⁶ to about 5×10⁶.

In some embodiments, methods of increasing an immune response in a subject in need thereof, is provided. In some embodiments, the methods comprise administering an effective amount of any of the cells provided herein. In some embodiments, the increased immune response is against a target antigen. In some embodiments, the increased immune response is humoral and/or cellular immune response. In some embodiments, the increased immune response is an increased in NK cells. In some embodiments, the increased immune response is an increased in NKT cells. In some embodiments, the increased immune response is an increase in activated NK cells. In some embodiments, the increased immune response is an increase in activated B cells. In some embodiments, the increased immune response is an increase in activated CD8 T cells. In some embodiments, the increased immune response is an increase in activated T cells as measured by percent of CD3+ and CD38+ cells. In some embodiments, the increased immune response is an increase in activated T cells as measured by percent of CD3+ and CD25+ cells. In some embodiments, the target antigen is a HIV protein. In some embodiments, the HIV protein is one or more of HIV Tat (full length or isoforms of 72 and 101 amino acids in length), Rev, Pol, GP120, GP160, GP41, env, Gag, Gag-Pol, Nef, Vpr, Vpu, or Vif, or any combination thereof. In some embodiments, the target antigen is expressed as the entire HIV genome, such as a from a heterologous nucleic acid molecule. In some embodiments, the HIV genomic nucleic acid comprises a mutation in the retroviral reverse transcriptase, and further wherein the HIV genomic nucleic acid does not encode a retroviral packaging signal, creating a disabled HIV genomic construct. In some embodiments, the HIV genome does not produce a HIV viral particle capable of replication. In some embodiments, the HIV genome does not produce a HIV viral particle capable of infecting a T-cell. In some embodiments, the cells are allogeneic to the subject. In some embodiments, the cells are not HLA-matched with the patient. In some embodiments, the dosage of cells is from about 1×10⁶ to about 1×10⁶ cells. In some embodiments, the immune response is against a viral infection, wherein the viral infection can be a human immunodeficiency virus (HIV) infection.

In some embodiments, the method comprises administering the composition more than once. In some embodiments, the compositions are administered from once every week, to once every 2 weeks, to once every 3 weeks, to once per month, to once every two months, to once every 3 months, to once every 4 months, to once every 5 months, to once every 6 months, or once every 7 months, or once every 8 months, or once every 9 months, or once every 10 months, or once every 11 months, or once annually as a maintenance treatment. The composition can, for example, be administered for as long as the subject exhibits improvement, decreased or undetectable viral titer, or stable/non-progressing disease that is being treated.

As cells, such as, for example recombinant allogeneic or autologous CD430 HIV vaccine cells or compositions comprising such cells may be used for cell therapy. The cells may be from a donor, or may be cells obtained from the patient. The cells may, for example, be used in regeneration, for example, to replace the function of diseased cells. The cells may also be modified to express a heterologous gene so that biological agents may be delivered to specific microenvironments such as, for example, diseased bone marrow or metastatic deposits. Mesenchymal stromal cells have also, for example, been used to provide immunosuppressive activity, and may be used in the treatment of graft versus host disease and autoimmune disorders.

In other examples, recombinant allogeneic or autologous CD4+ HIV vaccine cells or compositions comprising such cells are used to treat various diseases and conditions.

The term “unit dose” as it pertains to the inoculum refers to physically discrete units suitable as unitary dosages for mammals, each unit containing a predetermined quantity of pharmaceutical composition calculated to produce the desired immunogenic effect in association with the required diluent. The specifications for the unit dose of an inoculum are dictated by and are dependent upon the unique characteristics of the pharmaceutical composition and the particular immunologic effect to be achieved.

An effective amount of the pharmaceutical composition, comprising the recombinant allogeneic or autologous CD4+ HIV vaccine cells or compositions comprising such cells, would be the amount, such that over 60%, 70%, 80%, 85%, 90%, 95%, or 97% of the HIV infected cells are killed. The term is also synonymous with “sufficient amount.”

The effective amount for any particular application can vary depending on such factors as the disease or condition being treated, the particular composition being administered, the size of the subject, and/or the severity of the disease or condition. One can empirically determine the effective amount of a particular composition presented herein without necessitating undue experimentation.

The terms “contacted” and “exposed,” when applied to a cell, tissue or organism, are used herein to describe the process by which the pharmaceutical composition and/or another agent, such as for example a chemotherapeutic or radiotherapeutic agent, are delivered to a target cell, tissue or organism or are placed in direct juxtaposition with the target cell, tissue or organism. To achieve cell killing or stasis, the pharmaceutical composition and/or additional agent(s) are delivered to one or more cells in a combined amount effective to kill the cell(s) or prevent them from dividing. The administration of the pharmaceutical composition may precede, be co-current with and/or follow the other agent(s) by intervals ranging from minutes to weeks. In embodiments where the pharmaceutical composition and other agent(s) are applied separately to a cell, tissue or organism, one would generally ensure that a significant period of time did not expire between the times of each delivery, such that the pharmaceutical composition and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) with the pharmaceutical composition. In other aspects, one or more agents may be administered within of from substantially simultaneously, about 1 minute, to about 24 hours to about 7 days to about 1 to about 8 weeks or more, and any range derivable therein, prior to and/or after administering the expression vector. Yet further, various combination regimens of the pharmaceutical composition presented herein and one or more agents may be employed.

### Formulations and Routes for Administration to Patients

Where clinical applications are contemplated, it will be necessary to prepare pharmaceutical compositions--expression constructs, expression vectors, fused proteins, transfected or transduced cells, in a form appropriate for the intended application. Generally, this will entail preparing compositions that are essentially free of pyrogens, as well as other impurities that could be harmful to humans or animals.

The recombinant allogeneic or autologous CD4+ HIV vaccine cells or compositions comprising such cells, may be delivered, for example at doses of about 1-5 million cells per dose. Vials or other containers may be provided containing the recombinant cells at, for example, a volume per vial of about 0.25 ml to about 10 ml, for example, about 0.25, 0.5, 1, 1.5, 2, 2.5, 3, 3.5, 4, 4.5, 5, 5.5, 6, 6.5, 7, 7.5, 8, 8.5, 9, 9.5, or 10 ml, for example, about 2 ml.

One may generally desire to employ appropriate salts and buffers when recombinant cells are introduced into a patient. The phrase “pharmaceutically or pharmacologically acceptable” refers to molecular entities and compositions that do not produce adverse, allergic, or other untoward reactions when administered to an animal or a human. A pharmaceutically acceptable carrier includes any and all solvents, dispersion media, coatings, antibacterial and antifungal agents, isotonic and absorption delaying agents and the like. The use of such media and agents for pharmaceutically active substances is known. Except insofar as any conventional media or agent is incompatible with the vectors or cells, its use in therapeutic compositions is contemplated. Supplementary active ingredients also can be incorporated into the compositions.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution may be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media can be employed. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations may meet sterility, pyrogenicity, and general safety and purity standards as required by FDA Office of Biologics standards.

Additionally, in certain patients, it is expected that this treatment would be repeated periodically to boost the immune system response to any remaining virus/virions. Such periodic treatment can vary from once every week, once every 2 weeks, once every 3 weeks, once a month, to once every two months, to once every 3 months, to once every 4 months, to once every 5 months, to once every 6 months, or once every 7 months, or once every 8 months, or once every 9 months, or once every 10 months, or every 11 months, or once annually as a maintenance treatment for as long as the patient requires to achieve stable or undetectable disease.

In some embodiments, provided herein is an isolated cell transfected or transduced with a nucleic acid comprising a nucleotide sequence encoding CD40L and CXCL13.

In some embodiments, the CD40L comprises an amino acid sequence having at least 90% sequence identity to SEQ ID NO: 2, and wherein the CXCL13 comprises an amino acid sequence having at least 90% sequence identity to SEQ ID NO: 1.

In some embodiments, the cell is a T cell.

In some embodiments, the cell is transduced or transfected with a second and/or third nucleic acid that encodes a heterologous protein.

In some embodiments, the second nucleic acid comprises human immunodeficiency virus (HIV) genome, and wherein the HIV genomic nucleic acid comprises a mutation in the retroviral reverse transcriptase, and further wherein the HIV genomic nucleic acid does not encode a retroviral packaging signal, creating a disabled HIV genomic construct.

In some embodiments, provided herein is a CD4+ cell comprising one or more heterologous nucleic acid molecules encoding for CD40L and CXCL13.

In some embodiments, the CD40L comprises an amino acid sequence having at least 90% sequence identity to SEQ ID NO: 2, and wherein the CXCL13 comprises an amino acid sequence having at least 90% sequence identity to SEQ ID NO: 1.

In some embodiments, provided herein is a CD4+ cell comprising a heterologous CD40L protein and heterologous CXCL13 protein.

In some embodiments, the CD4+ cell further comprises heterologous nucleic acid molecule comprising human immunodeficiency virus (HIV) genome, and wherein the HIV genomic nucleic acid comprises a mutation in the retroviral reverse transcriptase, and further wherein the HIV genomic nucleic acid does not encode a retroviral packaging signal, creating a disabled HIV genomic construct.

In some embodiments, provided herein is a method of treating HIV in a subject comprising administering to the subject a composition comprising administering an effective amount of any of the cells described herein above.

In some embodiments, provided herein is a method for increasing immune response in a subject in need thereof, comprising administering an effective amount of any of the cells described herein above.

In some embodiments, the cells are allogeneic to the subject.

In some embodiments, the cells are not HLA-matched with the patient.

In some embodiments, the dosage of cells ranges from about 1-5×10⁶.

In some embodiments, the viral infection is caused by human immunodeficiency virus (HIV).

In some embodiments, graft versus host disease (GVHD) is decreased or eliminated, while graft versus virus (GVV) is increased in the subject.

In some embodiments, the treatment or increasing the immune response is repeated periodically for time frames of from once every week, to once every 2 weeks, to once every 3 weeks, to once per month, to once every two months, to once every 3 months, to once every 4 months, to once every 5 months, to once every 6 months, or once every 7 months, or once every 8 months, or once every 9 months, or once every 10 months, or once every 11 months, or once annually as a maintenance treatment for as long as the subject exhibits improvement, decreased or undetectable viral titer, or stable/non-progressing disease.

In some embodiments, cellular and humoral immunity are induced in the subject.

In some embodiments, embodiments provided herein also include, but are not limited to:
1. A cell comprising a heterologous nucleic acid molecule comprising a nucleotide sequence encoding CD40L and CXCL13.
2. The cell of embodiment 1, wherein the CD40L comprises an amino acid sequence having at least 90% sequence identity to SEQ ID NO: 4, and wherein the CXCL13 comprises an amino acid sequence having at least 90% sequence identity to SEQ ID NO: 3.
3. The cell of embodiment 1, wherein the cell heterologously expresses CD40L and CXCL13.
4. The cell of embodiment 1, wherein the cell is an isolated cell.
5. The cell of embodiment 1, wherein the cell is a T cell, such as a CD4+ T cell.
6. The cell of embodiment 1, wherein the cell is transduced or transfected with a second and/or third nucleic acid that encodes a heterologous protein or target antigen.
7. The cell of embodiment 6, wherein the second nucleic acid comprises human immunodeficiency virus (HIV) genome, and wherein the HIV genomic nucleic acid comprises a mutation in the retroviral reverse transcriptase, and further wherein the HIV genomic nucleic acid does not encode a retroviral packaging signal, creating a disabled HIV genomic construct.
8. The cell of embodiment 6, wherein the target antigen is a HIV protein.
9. The cell of embodiment 8, wherein the HIV protein is HIV Tat (full length or isoforms of 72 and 101 amino acids in length), Rev, Pol, GP120, GP160, GP41, env, Gag, Gag-Pol, Nef, Vpr, Vpu, or Vif, or any combination thereof.
10. The cell of any one of embodiments 1-9, wherein the cell is a CD4+ T cell.
11. A CD4+ T-cell comprising one or more heterologous nucleic acid molecules encoding for an amino acid sequence having at least 90% sequence identity to SEQ ID NO: 3, and/or an amino acid sequence having at least 90% sequence identity to SEQ ID NO: 4.
12. The CD4+ T-cell of embodiment 11, wherein the cell expresses CD40L.
13. The CD4+ T-cell of embodiments 11 and 12, wherein the cell expresses CXCL13.
14. The CD4+ T-cell of embodiment 11, wherein the cell expresses CD40L and CXCL13.
15. The CD4+ T-cell of embodiment 11, wherein the cell is an isolated CD4+ T-cell.
16. A CD4+ T-cell comprising a heterologous CD40L protein and heterologous CXCL13 protein.
17. The CD4+ T-cell of embodiment 16, further comprising heterologous nucleic acid molecule comprising human immunodeficiency virus (HIV) genome, and wherein the HIV genomic nucleic acid comprises a mutation in the retroviral reverse transcriptase, and further wherein the HIV genomic nucleic acid does not encode a retroviral packaging signal, creating a disabled HIV genomic construct.
18. The CD4+ T-cell of embodiment 16, further comprising a heterologous nucleic acid molecule encoding a target antigen.
19. The CD4+ T-cell of embodiment 18, wherein the target antigen is a HIV protein.
20. The CD4+ T-cell of embodiment 19, wherein the HIV protein is HIV Tat (full length or isoforms of 72 and 101 amino acids in length), Rev, Pol, GP120, GP160, GP41, env, Gag, Gag-Pol, Nef, Vpr, Vpu, or Vif, or any combination thereof.
21. A method of treating HIV in a subject comprising administering to the subject a composition comprising administering an effective amount of any of the cells of any one of embodiments 1-20.
22. The method of embodiment 22, wherein the composition is a pharmaceutical composition.
23. The method of embodiment 21, wherein the composition is administered intravenously or by infusion.
24. The method of any one of embodiments 21-23, wherein the cells are allogeneic to, or not HLA-matched to the subject.
25. The method of any one of embodiments 21-23, wherein the cells are autologous to the subject.
26. The method of any one of embodiments 21-25, wherein, the dosage of cells in the composition is from about 1×10⁶ to about 5×10⁶.
27. A method for increasing immune response in a subject in need thereof, comprising administering an effective amount of any of the cells of any one of embodiments 1-20.
28. The method of embodiment 27, wherein the increased immune response is against a target antigen.
29. The method of embodiment 28, wherein the target antigen is a HIV protein.
30. The method of embodiment 29, wherein the HIV protein is HIV Tat (full length or isoforms of 72 and 101 amino acids in length), Rev, Pol, GP120, GP160, GP41, env, Gag, Gag-Pol, Nef, Vpr, Vpu, or Vif, or any combination thereof.
31. The method of any of embodiments 27-29, wherein the cells are allogeneic to the subject.
32. The method of any of embodiments 27-29, wherein the cells are not HLA-matched with the patient.
33. The method of any of embodiments 27-32, wherein the dosage of cells ranges from about 1-5×10⁶.
34. The method of any of embodiments 27-33, wherein the immune response is against a viral infection, wherein the viral infection can be a human immunodeficiency virus (HIV) infection.
35. The method of any of embodiments 21-34, wherein the treatment or increasing the immune response is repeated periodically for time frames of from once every week, to once every 2 weeks, to once every 3 weeks, to once per month, to once every two months, to once every 3 months, to once every 4 months, to once every 5 months, to once every 6 months, or once every 7 months, or once every 8 months, or once every 9 months, or once every 10 months, or once every 11 months, or once annually as a maintenance treatment for as long as the subject exhibits improvement, decreased or undetectable viral titer, or stable/non-progressing disease.
36. The method of any of embodiments 21-35, wherein cellular and humoral immunity are induced in the subject.

The following examples are illustrative, but not limiting, of the compositions and methods described herein. Other suitable modifications and adaptations known to those skilled in the art are within the scope of the following embodiments.

### EXAMPLES

### Example 1

Constructing CD4+ Cells transduced with CD40L and CXCL13, and further loading with HIV genome

Step 1: Transduce CD4 cells (or other T cells) with lentivirus/adenovirus that over express CD40L and CXCL13 (B Cell attractant molecule) to produce a recombinant allogeneic CD4+ T cell expressing CD40L and CXCL13. This recombinant allogeneic CD4+ cell will function in the host to attract B cells to the area before the CD4 cells.

Step 2: Plasmid transfection and/or transposons delivery of HIV genome to the CD4+ CD40L+CXCL13+ cell. The CD4+ CD40L+CXCL13+ cell is loaded with incompetent HIV—replication incompetent or live attenuated genome. In preferred embodiments, the full HIV genome is utilized, wherein the reverse transcriptase (RT) comprises at least 1 mutation (or deletion) rendering it non-functional, and wherein there is further a mutation (or complete deletion) in the packaging signal (creating a replication incompetent HIV genome, but otherwise the full genome).

For creating the replication incompetent HIV genomic construct the following rationale and options are utilized—RT mutation and packaging signal mutation—
RT makes it infection incompetent
Packaging signal mutation—the CD4 would still produce virions if not have the packaging signal mutation. The recombinant CD4 cells would be budding out empty virions—envelope glycoprotein are part of the genome that induce neutralizing antibodies (creating a humoral response)
In certain embodiments, the construct can further include nucleic acids encoding different strains of different envelope proteins;
In certain embodiments, the construct can carry multiple variable envelope regions;

creating multiple glycoprotein (gp) structure—create diversity of the glycoproteins.
Thus, the CD4+ CD40L +CXCL13+ cell is loaded with an:
   HIV plasmid, with RT mutation, none or disabled packaging signal, multiple envelope proteins, creating a CD4+ cell expressing CD40L+CXCL13+ and expressing HIV envelope and glycoproteins to generate humoral immune response by the patient.
   Creating allogeneic CD4 cells as the HIV vaccine vehicle.

### Example 2

Evaluation of ENOB HV-11 and ENOB HV-12 in macaques as preventive and therapeutic vaccine candidates for HIV

As proof of concept, both human and non human primate (NHP) sequences of the 2 expression casettes (CD40L & CXCL13) will be tested using a lentiviral vector (LV) to investigate the bioactivity of, and provide proof-of-concept data for, ENOB HV-11 preventive HIV vaccine and ENOB HV-12 therapeutic HIV vaccine on non-human primates (macaque).

Allogeneic cells are a potent stimulus of an immune response. Allogeneic T-cells expressing HIV antigens genetically modified to express high levels of B-cell promoters CD40L and CXCL13 would be expected to induce a strong cellular and humoral reponse to be effective as a protective or therapeutic vaccine. Such recombinant allogeneic cells will be rapidly killed by the host immune system, and if provided in low enough numbers should not induce graph versus host dissease.

In examples described herein as proof of concept (POC), non human primates will be serially and subcutaneously injected with a few million (e.g. 1-5×10⁶) allogeneic T-cells genetically modified with either human or macaque sequence CD40L and CXCL13 that have been transfected with plasmid containing non-replicating, attenuated SHIV. Simian/human immunodeficiency virus (SHIV) is a series of chimeras created in laboratories whose genetic material is a combination of simian immunodeficiency virus (SIV) genes and HIV genes. It is capable of infecting almost every type of nonhuman primate that can be infected with SIV.

Neutralizing antibody titers will be measured. Once a protective level has been achieved, the animals will receive mucosal challenge with SHIV. If they are not protected from infection, they will be challenged intravenously. Any macaques that become infected, including the control cohort, will receive therapeutic vaccination with T-cells modified with the same casette (i.e. human versus macaques) infected with non-replicating, attenuated SHIV. The vector construct for HV-11 (for transducing human CXCL13 and human CD40L) is shown in FIG. 1A. The vector construct for HV-12 (for transducing macaque CXCL13 and macaque CD40L) is shown in FIG. 1B.

### Study Design Dosing and Schedule Details (See, FIGS. 2-4):

9 macaques; n=3 per group; potentially 2 stages of investigation spanning ENOB HV-11 and ENOB HV-12:
3 will receive completely MLA/HLA mismatched (CD4+) T-cells transfected with plasmid containing attenuated, replication incompetent SHIV and transduced with Human vector (cohort A) (FIG. 1A)
3 will receive completely mismatched CD4+ T-cells transfected with plasmid containing attenuated, replication incompetent SHIV and transduced with macaques vector (cohort B) (FIG. 1B)
3 will be the control group (no product received) (cohort C).

### ENOB HV-11 (FIG. 3)

Injection/Dosing/Evaluation schedule (cohorts A and B)
5 million cells/per week subcutaneously for 4 weeks; if there is reaction after the 1^{st} injection reduce the dose to 2 million cells/per week.
Following the 4^{th} injection, measure neutralizing antibody titer 7 days after the 4^{th}

Injection.
If the desired titer is not achieved, dose a second cycle with 2 million cells every 10 days for 4 shots s.c. , then, if needed, a 3^{rd} cycle of 2 million cells every 15 days for 4 shots s.c.
Once desired titer achieved, perform mucosal viral challenge SHIV. If mucosal challenge does not cause infection, administer intravenous challenge.
For each test subject, there will be weekly monitoring, antibody titer and safety labs.

ENOB HV-12 (FIG. 4)

Infected macaques from Cohorts A, B and C will undergo ART until then achieve viral suppression (<50 copies/ml) for 3 months. After achieving suppression, they will receive therapeutic vaccination with completely MLA mismatched CD4+ T-cells pulsed with replication incompetent, attenuated SHIV and matching vectors (i.e. Cohort A will receive human vector, and Cohort B will receive macaques vector) as described below and in FIGS. 2-4. An options is to utilize the same dosing regimen for HV-12, as was used for HV-11.

For Cohorts A and B, the T-cells will be from different, mismatched donors that the preventive vaccine.

Injection/Dosing/Evaluation schedule (cohort A, B and C)
5 million cells/per week subcutaneously for 4 weeks if there is reaction after the 1^{st} injection reduce the dose to 2 million cells/per week. An option is to start of dosing at peak viremia. (e.g. peak viremia in the n-3 control animals, no protective vaccine but challenged).
Measure plasma viremia 7 days after 4^{th} injection. If not <1 copy/ml, initiate subcutaneous injection of 2 million cells every 4 weeks
After 4^{th} (8^{th} total) injection measure plasma viremia. IF not <copy/ml, repeat cycle of 2 millions cells weekly for 4 weeks
Weekly monitoring for 6 months
Plasma viremia, lymphocyte subsets and safety labs
7 days following last subcutaneous injection of each cycle
plasma viremia, lymphocyte subsets, safety labs, GALT biopsy

### Follow-Up Period

1 year following last injection with either HV-11 or HV-12. Macaques will be sacrificed for full body evaluation of presence of SHIV and toxicity, e.g. lymphoma.

Toxicity monitoring during Proof of Concept (POC) study
Toxicities due to the ROA used.
Persistence of the product injected and associated expression.
Biodistribution of product injected.
Immune response directed against the product infused (humoral and cellular)
Tumorigenicity.
Macroscopic observations like weight and behavior.
Microscopic tissue pathologies.
Others

### Example 3

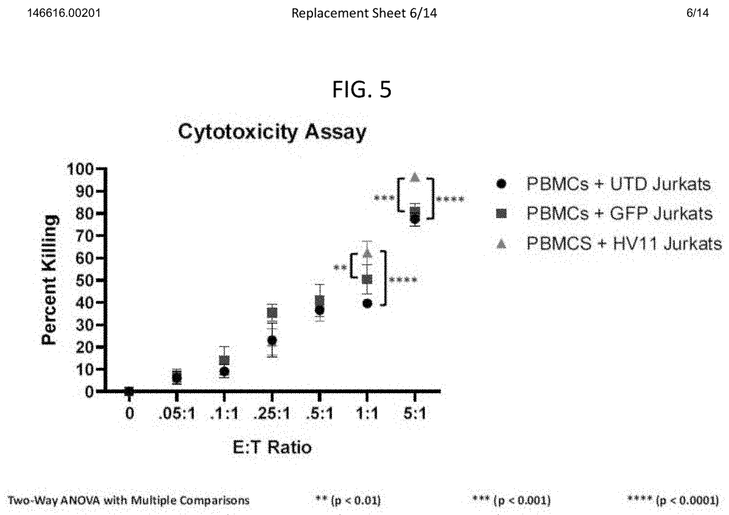

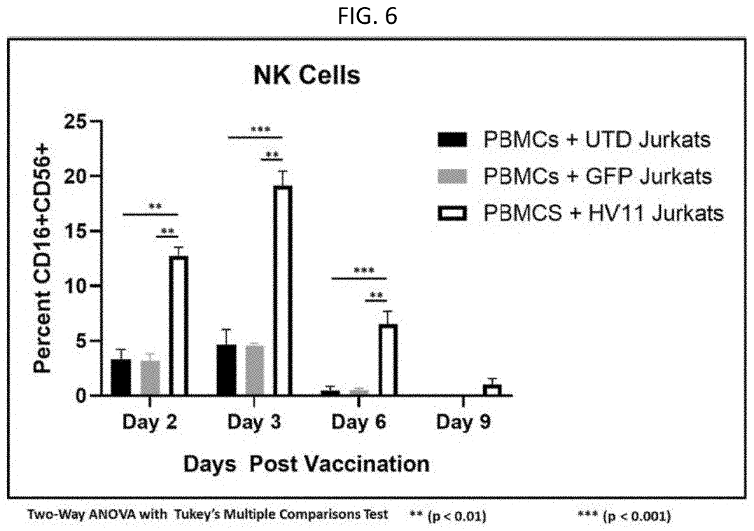

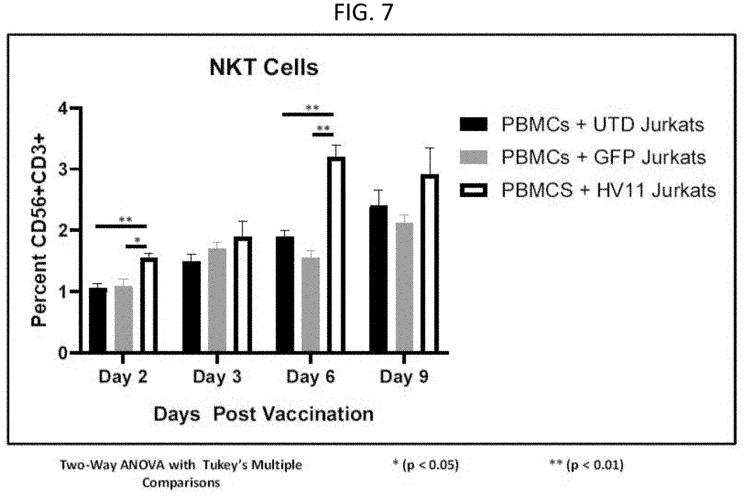

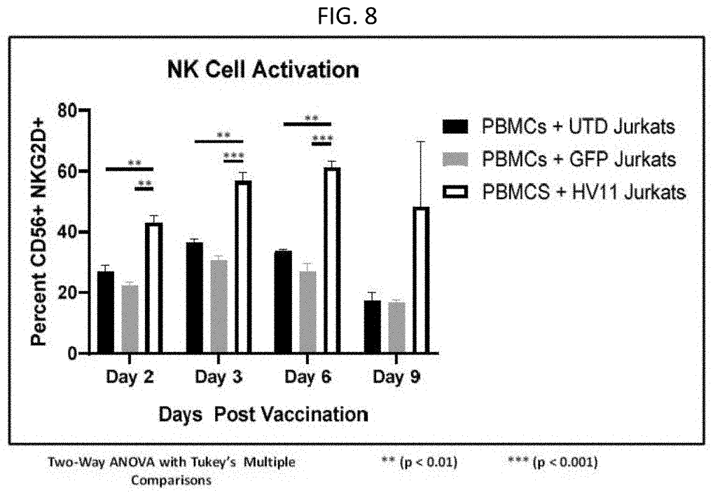

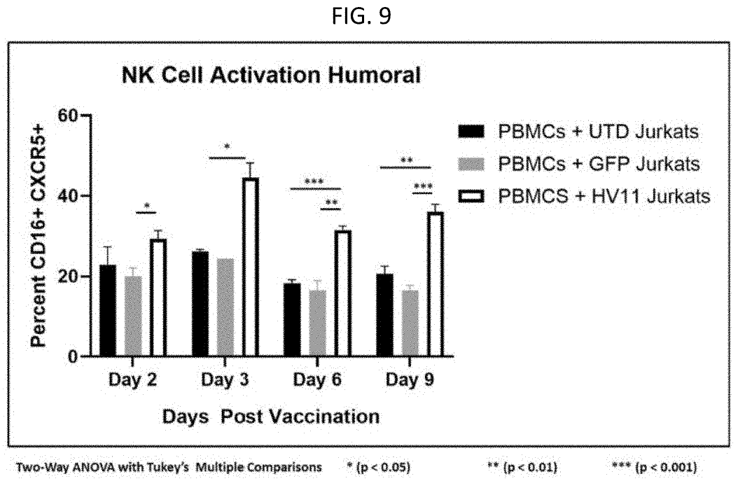

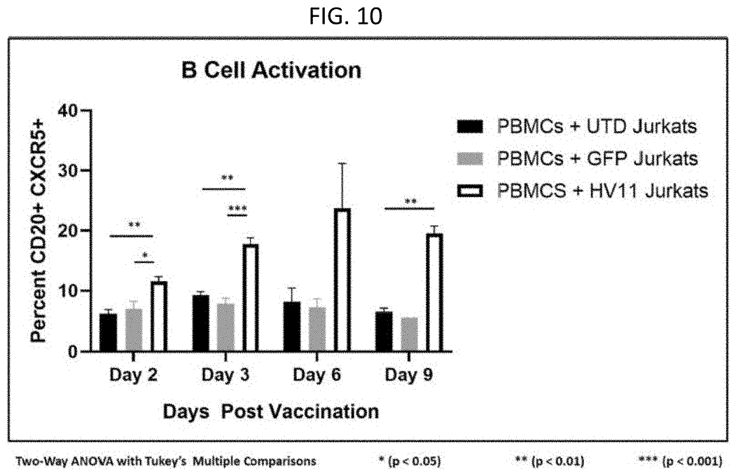

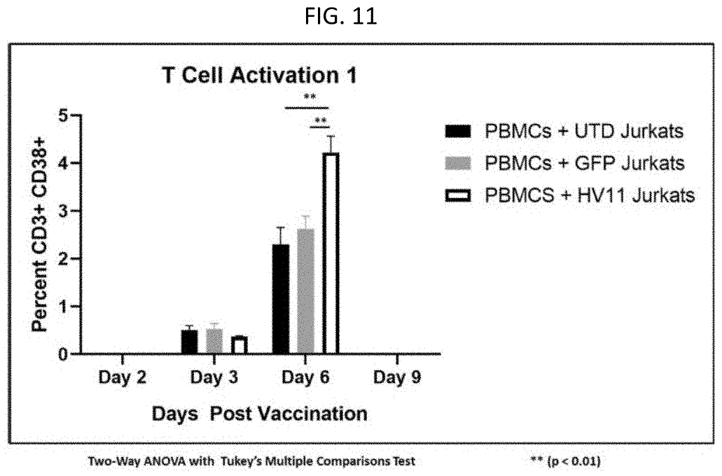

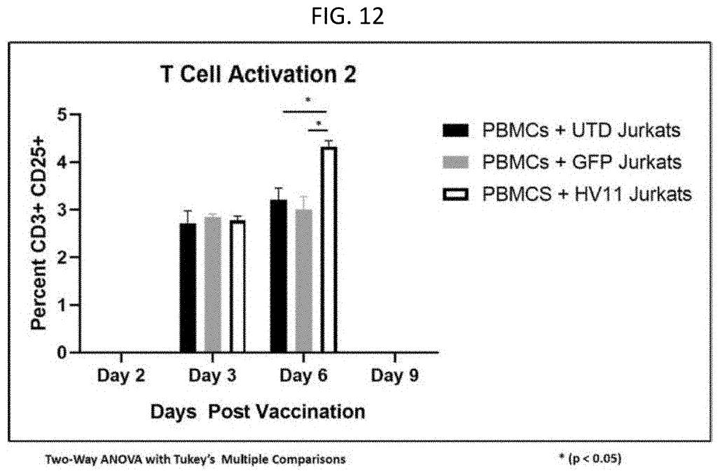

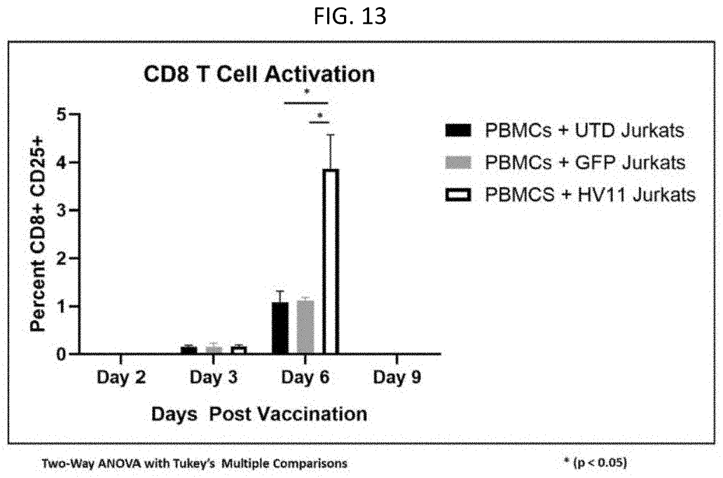

T cells transduced with CD40L and CXCL13 enhance cytotoxicity and increase immune cell activation. Vaccination combined with engineered allogenic effector cells expressing a target antigen, CD40L and CXCL13 enhances cytotoxic activity against the antigen. An in vitro model was developed to mimic in vivo cytotoxicity. A Jurkat-GFP expressing line was created to serve as the target for cytotoxicity to quantitatively measure specific killing activity of effectors. Normal donor PBMCs were then acquired for vaccination with three sets of cells to create a specific immune response against the Jurkat cells. Briefly, PBMCs are “vaccinated” with recombinant. (a) PBMCs were vaccinated with untransduced Jurkat cells (“UTD Jurkats”), (b) PBMCs were vaccinated with GFP transduced Jurkat cells (which serve as a nonspecific vector transduced control); and (c) PBMCs were vaccinated with Jurkat cells transduced with CD40L and CXCL13 (HV11). “Vaccination” in this example refers to the mixing of the PBMCs with the Jurkat cells mentioned above. This would be analogous to injecting recombinant CD4 T cells with CD40L and CXCL13 and injecting them into a patient with HIV or at risk of HIV, where the GFP is replaced with a HIV protein to be the training antigen for the PBMCs. After 9 days of PBMC vaccination, which allowed for expansion of Jurkat specific T cells, the Jurkat-GFP expressing target cells were co-cultured with the “vaccinated” PBMC cells (effector cells) for 18 hours to assay specific cytolytic activity. Cytolytic function was measured by flow cytometry (FACS) analysis to ascertain changes in GFP positive cells after co-culture. The data demonstrates that GFP expressing cells were killed more effectively with PBMCs that had been vaccinated with the T cells expressing GFP and CD40L and CXCL13 as compared to both untransduced Jurkats and GFP only-transduced Jurkat T cells. This data is illustrated in FIG. 5. The data show enhanced cytolytic function and was found to be dose dependent, i.e. as the PBMCs were vaccinated with increasing amounts of the CD40L and CXCL13 cells the cytotoxicity of the PBMCs increased. The effects on different types of immune cells were also measured. As illustrated in FIG. 6, the PBMCs vaccinated with the T cells heterologously expressing CD40L and CXCL13 saw a significant and substantial increase in NK cells (FIG. 6), NKT cells (FIG. 7), NK cell activation (FIG. 8), NK cell humoral activation (FIG. 9), B cell activation (FIG. 10), FIG. 10), T cell activation as measured by percent of CD3+ and CD38+ cells (FIG. 11), T cell activation as measured by percent of CD3+ and CD25+ cells (FIG. 12), and CD8 T cell activation (FIG. 13). The cells and activation were measured by flow cytometery using the surface markers indicated in the figures. Accordingly, these embodiments and data demonstrate the surprising and unexpected results of the ability to create a T cell that can enhance cytotoxicity against a specific antigen by having the T cell express the antigen along with CD40L and CXCL13, or active fragments thereof.

### Example 4

### Standard Methods

Standard methods in molecular biology are described Sambrook, Fritsch and Maniatis (1982 & 1989 2nd Edition, 2001 3rd Edition) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols.1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., N.Y., N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

All references cited herein are incorporated by reference to the same extent as if each individual publication, database entry (e.g. Genbank sequences or GenelD entries), patent application, or patent, was specifically and individually indicated to be incorporated by reference. This statement of incorporation by reference is intended by Applicants, pursuant to 37 C.F.R. §1.57(b)(1), to relate to each and every individual publication, database entry (e.g. Genbank sequences or GenelD entries), patent application, or patent, each of which is clearly identified in compliance with 37 C.F.R. §1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present embodiments are not to be limited in scope by the specific embodiments described herein. Indeed, various modifications of the invention in addition to those described herein will become apparent to those skilled in the art from the foregoing description and the accompanying figures. Such modifications are intended to fall within the scope of the embodiments provided herein and the appended claims.

1. A cell comprising a heterologous nucleic acid molecule comprising a nucleotide sequence encoding CD40L and CXCL13.

2. The cell of claim 1, wherein the CD40L comprises an amino acid sequence having at least 90% sequence identity to SEQ ID NO: 4, and wherein the CXCL13 comprises an amino acid sequence having at least 90% sequence identity to SEQ ID NO: 3.

3. The cell of claim 1, wherein the cell heterologously expresses CD40L and CXCL13.

4. The cell of claim 1, wherein the cell is an isolated cell.

5. The cell of claim 1, wherein the cell is a T cell.

6. The cell of claim 1, wherein the cell is transduced or transfected with a second and/or third nucleic acid that encodes a heterologous protein or target antigen.

7. The cell of claim 6, wherein the second nucleic acid comprises human immunodeficiency virus (HIV) genome, and wherein the HIV genomic nucleic acid comprises a mutation in the retroviral reverse transcriptase, and further wherein the HIV genomic nucleic acid does not encode a retroviral packaging signal, creating a disabled HIV genomic construct.

8. The cell of claim 6, wherein the target antigen is a HIV protein.

9. The cell of claim 8, wherein the HIV protein is one or more of HIV Tat (full length or isoforms of 72 and 101 amino acids in length), Rev, Pol, GP120, GP160, GP41, env, Gag, Gag-Pol, Nef, Vpr, Vpu, or Vif, or any combination thereof.

10. The cell of claim 6, wherein the cell0 is a CD4+ T cell.

11. A CD4+ T-cell comprising one or more heterologous nucleic acid molecules encoding for an amino acid sequence having at least 90% sequence identity to SEQ ID NO: 3, and/or an amino acid sequence having at least 90% sequence identity to SEQ ID NO: 4.

12-13. (canceled)

14. The CD4+ T-cell of claim 11, wherein the cell heterologously expresses CD40L and CXCL13.

15-20. (canceled)

21. A method of treating HIV in a subject comprising administering to the subject a composition comprising administering an effective amount of the cell of claim 8.

22. The method of claim 22, wherein the composition is a pharmaceutical composition.

23. (canceled)

24. The method of claim 21, wherein the cells are allogeneic to, or not HLA-matched to the subject.

25-26. (canceled)

27. A method for increasing immune response in a subject in need thereof, comprising administering an effective amount of the cell of claim 8.

28. The method of claim 27, wherein the increased immune response is against a target antigen.

29. The method of claim 28, wherein the target antigen is a HIV protein.

30. The method of claim 29, wherein the HIV protein is one or more of HIV Tat (full length or isoforms of 72 and 101 amino acids in length), Rev, Pol, GP120, GP160, GP41, env, Gag, Gag-Pol, Nef, Vpr, Vpu, or Vif, or any combination thereof.

31-36. (canceled)
